(19) United States
(12) Patent Application Publication
(10) Pub. No.: US 2007/0254928 A1
(43) Pub. Date: Nov. 1, 2007

(54) Use of Roflumilast for the Prophylaxis or Treatment of Emphysema

(75) Inventors: Stefan-Lutz Wollin, Meersburg (DE); Rolf Beume, Konstanz (DE); Giuseppe Lungarella, Siena (IT); Piero Martorana, Bad Homburg (IT)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/579,375

(22) PCT Filed: May 4, 2005
(86) PCT No.: PCT/EP05/52067

§ 371 Date: Dec. 6, 2006

Related U.S. Application Data
(60) Provisional application No. 60/569,462, filed on May 10, 2004.

(30) Foreign Application Priority Data
May 14, 2004 (EP) 04102136.1

(51) Int. Cl. A61K 31/44; A61P 11/00

(52) U.S. Cl. 514/352

(57) ABSTRACT

The invention relates to the use of roflumilast for the prophylaxis of or the treatment of emphysema.

### FIELD OF APPLICATION OF THE INVENTION

The present invention relates to the use of certain compounds for the prophylaxis of or for treating emphysema.

### BACKGROUND OF THE INVENTION

M-P. Pruniaux et al. describe in Am J Resp Crit Care Med 2003, Vol 167, A847 the efficacy of the selective phosphodiesterase 4 inhibitor CI-1044 on cigarette smoke-induced emphysema development in mice. K J. Stebbins et al describe in Am J Resp Crit Care Med 2003, Vol 167, A486 the aerosol activity of different PDE4 inhibitors in a murine model of cigarette smoke induced pulmonary inflammation. J C Fox et al describe in Am J Resp Crit Care Med 2003, Vol 167, A91 the efficacy of the PDE4 inhibitor BAY19-8004 in Tobacco Smoke Models of COPD in the guinea pig. In the international patent applications WO03039552 (US2003/092706) and WO03097050 the combination of PDE4 inhibitors with DMARDs respectively iNOS inhibitors is described; both combination applications mention as a possible indication emphysema. In the European patent application EP1199074 the use of a PDE4 inhibitor for preventing or treating a disease associated with an excess in IL-12 production is described; here too, emphysema is mentioned as a possible indication. In Bundschuh et al; JPET 297, no 1, 2001, pp 280-290 the in vivo efficacy of Roflumilast in airway disease models is described.

### DESCRIPTION OF THE INVENTION

Emphysema is a condition in which there is over-inflation of structures in the lungs known as alveoli or air sacs. This over-inflation results from the breakdown of the walls of the alveoli, which causes a decrease in respiratory function and often, breathlessness. Early symptoms of emphysema include shortness of breath and cough.

According to the lung disease data report 2003 published by the American Lung Association an estimated 3 million Americans have been diagnosed with emphysema—close to 1.7 million males and 1.3 million females. Classic emphysema develops over many years of assault on lung tissues. The walls between the tiniest air sacs within the lungs break down, and those compartments become unnaturally enlarged. Elasticity of the lung tissue is lost, and the lungs become distended, unable to inflate and deflate normally. As emphysema progresses, the effort needed to breathe increases and, ultimately, each breath becomes a chore. Meanwhile, the patient grows progressively short of breath—at first experiencing only minimal shortness of breath, soon unable to attempt even minor physical activity, and in the end dependent on continuous administration of oxygen. The damage and the disease are regarded as irreversible. Normally, therapy is limited to relief of symptoms and attempts to improve the patient's quality of life.

Emphysema most commonly is caused by smoking. Stopping smoking is therefore the single most important way of affecting outcome in patients at all stages of emphysema. Currently used medications include bronchodilators, which are used to help open the airways in the lungs and decrease shortness of breath. Inhaled or oral steroids are used to help decrease inflammation in the airways in some people. Antibiotics are often used to treat additional infections; expectorants are sometimes used to help clear mucus from the airways. All these medications can help control, but not cure, emphysema.

Therefore, there is a high need for further medicaments for the prophylaxis of or for the treatment of emphysema.

It has now been found that roflumilast is useful, in addition to previously mentioned indications, for the prophylaxis of or the treatment of emphysema.

The invention thus relates in a first aspect to the use of roflumilast in the production of a pharmaceutical composition for the prophylaxis of or the treatment of emphysema.

In a second aspect the invention relates to a method for the prophylaxis of or the treatment of emphysema in a patient comprising administering to said patient a therapeutically effective amount of roflumilast.

In a third aspect the invention relates to a method for the prophylaxis of or the treatment of emphysema in a patient comprising administering to said patient a therapeutically effective amount of roflumilast in a free or fixed combination with an effective amount of a member selected from the group of β₂ adrenoceptor agonists, particularly long acting β₂ adrenoceptor agonists such as salmeterol, formoterol or (R,R)-formoterol, and pharmaceutically acceptable salts thereof, steroids, e. g. budesonide, fluticasone, flunisolide, beclomethasone, mometasone, methyl prednisolone and ciclesonide, and pharmaceutically acceptable salts thereof, and anticholinergic agents, e. g. oxytropium, ipratropium and tiotropium salts, in particular the bromide salts thereof.

As mentioned above, pulmonary emphysema causes progressive destruction of lung tissue, eventually resulting in respiratory failure. The primary target of tissue injury appears to be elastic fibers, which are degraded by elastases that accumulate in the lung as a result of cigarette smoking, air polluants, infections and other factors.

The elastic fibers that undergo breakdown in pulmonary emphysema have a highly specialized structure, consisting of an amorphous core elastin protein surrounded by layers of microfibrils. The elastin protein is composed of a network of polypeptides joined together by the coalescence of lysine side-chains into crosslinking structures, particularly desmosine and isodesmosine.

The increased breakdown of the elastic fibers results in a decrease of lung desmosine and isodesmosine content and an increase of the urine levels of desmosine and isodesmosine. Urine levels of desmosine and isodesmosine are therefore considered representative of elastin breakdown. Studies have shown that urinary desmosine excretion is significantly higher in patients with chronic obstructive pulmonary disease than in healthy controls. In COPD patients with no evidence or only mild emphysema, desmosine excretion values were significantly higher than those of patients with moderate to severe emphysema, due to the depletion of elastin, the source of desmosine, in the moderate to severe emphysema patients.

In a chronic model of cigarette smoke induced emphysema in mice it was shown that the administration of Roflumilast can prevent the drop of the desmosine content in the lung.

In a fourth aspect the invention therefore relates to a method for the prevention or reduction of the lung desmosine content decrease in a patient comprising administering to said patient a therapeutically effective amount of roflumilast.

As mentioned above, the decrease of the lung desmosine and isodesmosine content leads to an increase of the urine levels of desmosine and isodesmosine.

In a fifth aspect the invention relates to a method for the treatment of mild emphysema in a patient comprising
(a) determination of the amount of desmosine in the urine of said patient
(b) comparing the amount of desmosine in the urine of said patient with the amount of desmosine in the urine of a healthy subject, and in case the amount of desmosine in the urine of said patient is higher than the amount of desmosine in the urine of a healthy subject,
(c) administering to said patient a therapeutically effective amount of roflumilast.

The amount of desmosine in the urine can be determined by different methods, for example by the indirect competitive enzyme-linked immunosorbent assay described in Franca Cocci et al; International Journal of Biochemistry & Cell Biology 2002 Vol 34, pp 594-604, by a high-performance capillary electrophoresis method described in Viglio S et al; European Respiratory Journal 2000 Vol 15, pp 1039-1045, or by a modified radioimmunoassay described by Starcher et al; Respiration 1995; Vol 62, pp 252-257.

Another useful indicator for the degree of emphysema is the determination of the mean linear intercept (Lm), i. e. the mean distance between alveolar walls on 10 parallel transverse lines drawn in each examined field (Thurlbeck et al. Am Rev Respir Dis 1967; 95: 752-64). Studies with cigarette smoke induced emphysema mice models have shown that Lm is significantly increased in cigarette smoke exposed mice compared to sham animals.

Here too, it was shown in a chronic model of cigarette smoke induced emphysema in mice that the administration of Roflumilast can prevent the increase of Lm.

In a sixth aspect the invention therefore relates to a method for the prevention or reduction of an increase of the average inter-alveolar distance [mean linear intercept (Lm)] in a patient comprising administering to said patient a therapeutically effective amount of roflumilast.

The mean linear intercept (Lm) may be determined by high-resolution computerized tomography (HRCT).

In the sense of the invention, the term “roflumilast” is understood to include ROFLUMILAST, the pharmaceutically acceptable salts of ROFLUMILAST, the N-oxide of ROFLUMILAST and the pharmaceutically acceptable salts of the latter, which can likewise be used according to the invention.

ROFLUMILAST is the international non proprietary name (INN) for 3-cyclopropylmethoxy-4-difluoro-methoxy-N-(3,5-dichloropyrid-4-yl)benzamide [structure of formula (1.1)]. The preparation of 3-cyclopropylmethoxy-4-difluoromethoxy-N -(3,5-dichloropyrid-4-yl)benzamide, its pharmaceutically acceptable salts and its N-oxide [3-cyclopropylmethoxy-4-difluoromethoxy-N-(3,5-dichloro-1-oxido-pyrid-4-yl)-benzamide; [structure of formula (1.2)] as well as the use of these compounds as phosphodiesterase (PDE) 4 inhibitors is described in WO95/01338.

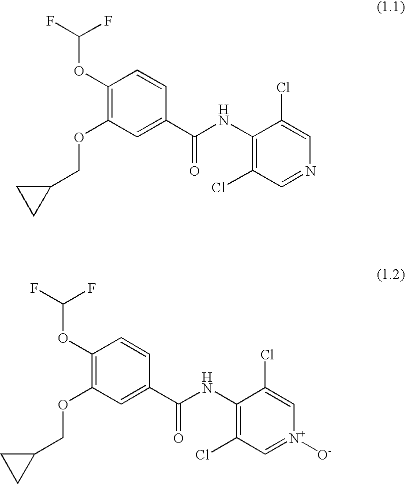

Salts encompassed within the term “pharmaceutically acceptable salts” refer to non-toxic salts of the compounds which are generally prepared by reacting a free base with a suitable organic or inorganic acid or by reacting an acid with a suitable organic or inorganic base. Particular mention may be made of the pharmaceutically acceptable inorganic and organic acids customarily used in pharmacy. Those suitable are in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid. As examples of pharmaceutically acceptable salts with bases may be mentioned the lithium, sodium, potassium, calcium, aluminium, magnesium, titanium, ammonium, meglumine or guanidinium salts.

It is understood that the active compounds and their pharmaceutically acceptable salts mentioned can also be present, for example, in the form of their pharmaceutically acceptable solvates, in particular in the form of their hydrates.

Roflumilast may be administered to a patient in need of treatment in any of the generally accepted modes of administration available in the art. Illustrative examples of suitable modes of administration include oral, intravenous, nasal, parenteral, transdermal and rectal delivery as well as administration by inhalation. The most preferred mode of administration of roflumilast is oral. In another preferred embodiment roflumilast is administered by intravenous infusion or injection.

Pharmaceutical compositions are prepared by processes which are known per se and familiar to the person skilled in the art. As pharmaceutical composition, roflumilast (=active compound) is either employed as such, or preferably in combination with suitable pharmaceutical auxiliaries and/or excipients, e.g. in the form of tablets, coated tablets, capsules, caplets, suppositories, emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

Suitable oral dosage forms of roflumilast are described in the international patent application WO03/070279.

Roflumilast can also be administered in the form of an aerosol; the aerosol particles of solid, liquid or mixed composition preferably having a diameter of 0.5 to 10 μm, advantageously of 2 to 6 μm. Aerosol generation can be carried out, for example, by pressure-driven jet atomizers or ultrasonic atomizers, by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

It is known to the person skilled in the art that the optimum dose of an active compound can vary as a function of body weight, the age and the general condition of the patient, and his/her response behaviour to the active compound.

In case of oral administration of 3-cyclopropylmethoxy-4-difluoromethoxy-N-(3,5-dichloropyrid-4-yl)-benzamide (ROFLUMILAST), the adult daily dose is in the range from 50-1000 μg, preferably in the range from 250-500 μg, preferably by once daily administration.

In case of intravenous administration of 3-cyclopropylmethoxy-4-difluoromethoxy-N-(3,5dichloropyrid-4-yl)benzamide (ROFLUMILAST), the adult daily dose is in the range from 50-600 μg, preferably in the range from 150-300 μg.

Pharmacology

Effects of ROFLUMILAST in an Chronic Model of Cigarette Smoke Exposure in Mice

Animals

Eighty 6 week old male mice of the strain C57BI/6J (supplied Harlan-Italy, Udine, Italy) were used in this study. The mice were housed in groups of 7 to 10 in macrolon cages. Room temperature was kept at 22° to 24° C.; and relative humidity at 40 to 50%; food and water were supplied ad libitum. All animal experimentation was approved by the Local Ethical Committee of the University of Siena.

ROFLUMILAST Treatment

Five groups of 10-20 animals each were made as follows:

| | |
|---|---|
| 1) No treatment/air exposed | N = 13 |
| 2) ROFLUMILAST 5 mg/kg/air exposed | N = 15 |
| 3) No treatment/smoke exposed | N = 15 |
| 4) ROFLUMILAST 1 mg/kg/smoke exposed | N = 20 |
| 5) ROFLUMILAST 5 mg/kg/smoke exposed | N = 20 |

ROFLUMILAST was given p.o. by gavage in a volume of 10 μl suspension/g body weight 60 min prior to either air or smoke exposure.

For 1 mg/kg use 10 mg of ROFLUMILAST was suspended in 100.3 ml of a 4% methocel solution (Methylhydroxypropyl Cellulose 2910.15 CPS, Dow Chemicals, Mideland, Md., USA), containing 1.3 ml polyethylene glycol 400 (Merck-Schuchhard, Hohenheim, Germany) and two drops (about 50 μl) of Antifoam C (Simethicon emulsion 30%). The suspension was stirred with ultra-thurrax for 10 minutes. This suspension (stable at 4° C. for one week) was agitated with a magnetic stirrer before use.

For 5 mg/kg use 75 mg of ROFLUMILAST was suspended in 150 ml of 4% methocel solution, containing 2 ml of PEG, and two drops of Antifoam C. This suspension was stirred with ultra-thurrax for 10 minutes and agitated with a magnetic stirrer before use.

Chronic Exposure to Cigarette Smoke

The methodology for chronic smoke exposure has been previously described in detail (Cavarra et al; Am J Respir Crit Care Med 2001; Vol 164, pp 886-890). Briefly, mice were exposed to either the smoke of 3 cigarettes/day (commercial Virginia filter cigarettes: 12 mg of tar and 0.9 mg of nicotine), 5 days/week or to room air (controls) for 7 months, in especially designed macrolon cages (Tecniplast, Buguggiate, Italy). These cages (42.5×26.6×19 cm) are equipped with a disposable filter cover which enables the air to flow out of the cages and thus to be continuously renewed. The smoke was produced by the burning of a cigarette and was introduced into the chamber with the airflow generated by a mechanical ventilator (7025 Rodent Ventilator, Ugo Basile, Biological Research Instruments, Comerio, Italy), at a rate of 33 ml /min. A second mechanical ventilator was used to provide room air for dilution (1:8) of the smoke-stream. With this methodology the mice were exposed to the smoke originated by three cigarettes once a day for the duration of 90 min. In a pilot study, the efficiency of the smoke delivery system was tested in 12 mice by measuring blood HbCO by co-oxymetry.

Light Microscopy: Morphology, Morphometry

Seven months after chronic exposure to room air or cigarette smoke (24 h after last exposure), 5 to 12 animals of each group, were anesthetized with ether and then exsanguinated by severing the abdominal aorta. The lungs were excised and fixed intratracheally with buffered formalin (5%) at a constant pressure of 20 cm H₂O for at least 24 hours. Post-fixation lung volume (V_{L}) was measured by water displacement. All lungs were then dehydrated, cleared in toluene and embedded under vacuum in paraffin. Two 7-μm transversal sections were made and stained with hematoxylin-eosin and/or periodic acid-Schiff (PAS). Two pathologists blinded to the exposure protocol carried out the morphological and morphometrical evaluation.

Emphysema

Morphometric assessment of emphysema included determination of the average inter-alveolar distance (mean linear intercept: Lm) and internal surface area estimated by the Lm method at postfixation lung volume. For the determination of the Lm for each pair of lungs, 40 histological fields were evaluated both vertically and horizontally. Examination of these numbers of fields meant that practically the entire lung area was evaluated. Internal surface area of the lung (ISA) was calculated by according to the formula: ISA=4V_{L}/Lm.

Biochemistry

Lung Desmosine Content-Preparation of Lung Samples

Lung desmosine concentration was determined by High Pressure Liquid Chromatography (HPLC) essentially according to Cumiskey et al. (J Chromatogr B 1995, Vol 668, pp 199-207). Briefly, lung samples were homogenized in 5% TCA (1:9, w:v) and centrifuged for 10 min. at 4000 g at 4° C. The pellet was then washed twice with distilled water and hydrolyzed for 16 h at 130° C. in 6 N HCl.

After hydrolysis, the samples were centrifuged for 10 min at 2000 g and filtered through a FP 030/3 0.2 μm filter (Schleicher & Schuell). Aliquots (0.5 ml) of samples were desiccated under liquid nitrogen and then suspended in 0.6 ml 0.1 M sodium phosphate, pH 3.73.

HPLC Analysis of Processed Biological Samples

The HPLC apparatus consisted of a single pump (Pro-Star 210, Varian) delivering isocratic mobile phase, of which 80% was 0.1 M dibasic sodium phosphate adjusted to pH 3.75 with phosphoric acid; the remaining 20% was acetonitrile. Sodium dodecyl sulphate (SDS) was added to a final concentration of 10 mM. The pH was re-adjusted after the addition of acetonitrile and SDS. The flow rate was 0.8 ml/min through a C18 (reverse phase MSORV 100A, Varian). Injection volumes of 100 μl of test samples diluted in 0.1 M sodium phosphate buffer, pH 3.75 was used to detect desmosine concentration. Detection of desmosine was by absorbance at 275 nm. Peak purity was checked with a Pro-Star 330 photodiode array detector (Varian) scanning from 200 to 400 nm. Isodesmosine eluted at approx. 9 min, and desmosine approx. at 12 min. Peak height, appopriate external calibration curves and internal standards were used to quantitate desmosine in unknown samples. Desmosine standards were from Elastin Co. (USA).

Statistical Analysis

All data were analyzed using GraphPad Prism software (GraphPad Software, San Diego, Calif., USA). For each parameter the values of the individual animals were averaged and the SEM was calculated. The significance of the differences was calculated using parametric one-way analysis of variance (ANOVA) with subsequent Bonferroni's multiple comparison post-test for selected pairs. A p value of <0.05 was considered significant.

Results

Mortality

No animal died in the course of the study.

Morphometric analysis of 3 animals of the ROFLUMILAST 5 mg/kg smoke exposure group was not conducted due to technical reasons.

Emphysema

Data given in table 1, revealed that the lungs of smoke exposed, untreated animals were significantly different from air exposed. This means that chronic cigarette smoke exposure induced significant emphysematous alveolar space enlargement with increase of Lm and decrease of ISA. Similar changes were seen in the smoke-exposed animals, which were treated with ROFLUMILAST 1 mg/kg, showing that the low dose of ROFLUMILAST was unable to inhibit emphysema formation. However, air-exposed, air-exposed and ROFLUMILAST 5 mg/kg treated, and smoke-exposed ROFLUMILAST 5 mg/kg treated animals all had similar values with regard to both the Lm and ISA, indicating gross inhibition of emphysema formation by ROFLUMILAST.

TABLE 1
Effect of ROFLUMILAST at two doses on Chronic Cigarette Smoke Exposure
(Emphysema)

| Group | N | Lm (μm) | ISA (cm²) |
|---|---|---|---|
| Air exposure | 5 | 33.54 ± 0.31### | 1305.57 ± 81.08# |
| Air exposure + R5 | 8 | 33.48 ± 0.36### | 1342.70 ± 23.39## |
| Cigarette smoke exposure | 8 | 40.69 ± 0.70*** | 1130.31 ± 25.96* |
| Cigarette smoke exposure + R1 | 12 | 40.79 ± 0.87*** | 1079.78 ± 35.61** |
| Cigarette smoke exposure + R5 | 9 | 34.28 ± 0.31### | 1292.21 ± 29.63# |

Data are given as mean ± SEM.
Abbreviations: N= number of animals;
Lm = mean linear intercept;
ISA = internal surface area of the lung;
R1 = ROFLUMILAST at the dose of 1 mg/kg;
R5 = ROFLUMILAST at the dose of 5 mg/kg
* = p <0.05,
** = p <0.01,
*** = p <0.001 versus “Air exposure”
# = p < 0.05,
## = p < 0.01,
### = p < 0.001 versus “Cigarette smoke exposure”

Lung Desmosine Content

The result of the assessment of the lung desmosine content in the various groups are shown in Table 2. The lungs of smoke-exposed animals as well as the lungs of smoke-exposed and ROFLUMILAST 1 mg/kg treated animals showed significantly lower lung desmosine content compared to the lungs of air-exposed and air-exposed and ROFLUMILAST 5 mg/kg treated animals, reflecting cigarette smoke induced elastolytic, proteolytic destruction of the parenchyma and alveolar walls. Unchanged lung desmosine content in the smoke-exposed and ROFLUMILAST 5 mg/kg treated group compared to air-exposure reflects gross inhibition of lung parenchyma and alveoli destruction by cigarette smoke exposure in animals treated with ROFLUMILAST. These results perfectly match the results of the morphometrical assessment of emphysema.

TABLE 2
Effect of ROFLUMILAST at two doses on Chronic Cigarette
Smoke Exposure (Lung Desmosine).

| Group | N | Desmosine (μg/lung) |
|---|---|---|
| Air exposure | 8 | 2.89 ± 0.07# |
| Air exposure + R5 | 7 | 2.91 ± 0.08# |
| Cigarette smoke exposure | 7 | 2.50 ± 0.11* |
| Cigarette smoke exposure + R1 | 8 | 2.45 ± 0.11* |
| Cigarette smoke exposure + R5 | 8 | 2.98 ± 0.10## |

Data are given as mean ± SEM.
Abbreviations: N = number of animals;
R1 = ROFLUMILAST at the dose of 1 mg/kg;
R5 = ROFLUMILAST at the dose of 5 mg/kg
* = p <0.05 versus “Air exposure”
# = p <0.05,
## = p <0.01 versus “Cigarette smoke exposure”

1.-3. (canceled)

4. A method for the prophylaxis of or the treatment of emphysema in a patient comprising administering to a patient in need thereof a therapeutically effective amount of roflumilast.

5. A method for the prophylaxis of emphysema in a patient comprising administering to a patient in need thereof a therapeutically effective amount of roflumilast.

6. A method for the treatment of emphysema in a patient comprising administering to a patient in need thereof a therapeutically effective amount of roflumilast.

7. A method for the prevention or reduction of lung desmosine content decrease in a patient comprising administering to a patient in need thereof a therapeutically effective amount of roflumilast.

8. A method for the treatment of mild emphysema in a patient comprising
(a) determining the amount of desmosine in a patient's urine
(b) comparing the amount of desmosine in the urine of said patient with the amount of desmosine in a healthy subject's urine, and in case the amount of desmosine in the urine of said patient is higher than the amount of desmosine in the urine of said healthy subject,
(c) administering to said patient a therapeutically effective amount of roflumilast.

9. A method for the prevention or reduction of an increase of the average inter-alveolar distance [mean linear intercept (Lm)] in a patient comprising administering to a patient in need thereof a therapeutically effective amount of roflumilast.

10.-13. (canceled)

14. The method according to claim 4, wherein roflumilast represents 3-cyclopropylmethoxy-4-difluoromethoxy-N-(3,5-dichloropyrid-4-yl)benzamide (ROFLUMILAST).

15. The method according to claim 4, wherein roflumilast represents 3-cyclopropylmethoxy-4-difluoromethoxy-N-(3,5-dichloro-1-oxido-pyrid-4-yl)benzamide (ROFLUMILAST-N-Oxide).

16. The method according to claim 4, wherein roflumilast represents a pharmaceutically acceptable salt of 3-cyclopropylmethoxy-4-difluoromethoxy-N-(3,5-dichloropyrid-4-yl)benzamide (ROFLUMILAST).

17. The method according to claim 4, wherein roflumilast represents a pharmaceutically acceptable salt of 3-cyclopropylmethoxy-4-difluoromethoxy-N-(3,5-dichloro-1-oxido-pyrid-4-yl)benzamide (ROFLUMILAST-N-Oxide).

18.-19. (canceled)

20. The method according to claim 14, wherein the daily therapeutically effective amount of 3-cyclopropylmethoxy-N-(3,5-dichloropyrid-4-yl)benzamide (ROFLUMILAST) for an adult patient is 500 μg.

21. The method according to claim 15, wherein the daily therapeutically effective amount of 3-cyclopropylmethoxy-N-(3,5-dichloro-1-oxido-pyrid-4-yl)benzamide (ROFLUMILAST-N-Oxide) for an adult patient is 500 μg.

22. The method according to claim 5, wherein roflumilast represents 3-cyclopropylmethoxy-4-difluoro-methoxy-N-(3,5-dichloropyrid-4-yl)benzamide (ROFLUMILAST).

23. The method according to claim 5, wherein roflumilast represents 3-cyclopropylmethoxy-4-difluoro-methoxy-N-(3,5-dichloro-1-oxido-pyrid-4-yl)benzamide (ROFLUMILAST-N-Oxide).

24. The method according to claim 5, wherein roflumilast represents a pharmaceutically acceptable salt of 3-cyclopropylmethoxy-4-difluoromethoxy-N-(3,5-dichloropyrid-4-yl)benzamide (ROFLUMILAST).

25. The method according to claim 5, wherein roflumilast represents a pharmaceutically acceptable salt of 3-cyclopropylmethoxy-4-difluoromethoxy-N-(3,5-dichloro-1-oxido-pyrid-4-yl)benzamide (ROFLUMILAST-N-Oxide).

26. The method according to claim 6, wherein roflumilast represents 3-cyclopropylmethoxy-4-difluoro-methoxy-N-(3,5-dichloropyrid-4-yl)benzamide (ROFLUMILAST).

27. The method according to claim 6, wherein roflumilast represents 3-cyclopropylmethoxy-4-difluoro-methoxy-N-(3,5-dichloro-1-oxido-pyrid-4-yl)benzamide (ROFLUMILAST-N-Oxide).

28. The method according to claim 6, wherein roflumilast represents a pharmaceutically acceptable salt of 3-cyclopropylmethoxy-4-difluoromethoxy-N-(3,5-dichloropyrid-4-yl)benzamide (ROFLUMILAST).

29. The method according to claim 6, wherein roflumilast represents a pharmaceutically acceptable salt of 3-cyclopropylmethoxy-4-difluoromethoxy-N-(3,5-dichloro-1-oxido-pyrid-4-yl)benzamide (ROFLUMILAST-N-Oxide).

30. The method according to claim 7, wherein roflumilast represents 3-cyclopropylmethoxy-4-difluoro-methoxy-N-(3,5-dichloropyrid-4-yl)benzamide (ROFLUMILAST).

31. The method according to claim 7, wherein roflumilast represents 3-cyclopropylmethoxy-4-difluoro-methoxy-N-(3,5-dichloro-1-oxido-pyrid-4-yl)benzamide (ROFLUMILAST-N-Oxide).

32. The method according to claim 7, wherein roflumilast represents a pharmaceutically acceptable salt of 3-cyclopropylmethoxy-4-difluoromethoxy-N-(3,5-dichloropyrid-4-yl)benzamide (ROFLUMILAST).

33. The method according to claim 7, wherein roflumilast represents a pharmaceutically acceptable salt of 3-cyclopropylmethoxy-4-difluoromethoxy-N-(3,5-dichloro-1-oxido-pyrid-4-yl)benzamide (ROFLUMILAST-N-Oxide).

34. The method according to claim 8, wherein roflumilast represents 3-cyclopropylmethoxy-4-difluoro-methoxy-N-(3,5-dichloropyrid-4-yl)benzamide (ROFLUMILAST).

35. The method according to claim 8, wherein roflumilast represents 3-cyclopropylmethoxy-4-difluoro-methoxy-N-(3,5-dichloro-1-oxido-pyrid-4-yl)benzamide (ROFLUMILAST-N-Oxide).

36. The method according to claim 8, wherein roflumilast represents a pharmaceutically acceptable salt of 3-cyclopropylmethoxy-4-difluoromethoxy-N-(3,5-dichloropyrid-4-yl)benzamide (ROFLUMILAST).

37. The method according to claim 8, wherein roflumilast represents a pharmaceutically acceptable salt of 3-cyclopropylmethoxy-4-difluoromethoxy-N-(3,5-dichloro-1-oxido-pyrid-4-yl)benzamide (ROFLUMILAST-N-Oxide).

38. The method according to claim 9, wherein roflumilast represents 3-cyclopropylmethoxy-4-difluoro-methoxy-N-(3,5-dichloropyrid-4-yl)benzamide (ROFLUMILAST).

39. The method according to claim 9, wherein roflumilast represents 3-cyclopropylmethoxy-4-difluoro-methoxy-N-(3,5-dichloro-1-oxido-pyrid-4-yl)benzamide (ROFLUMILAST-N-Oxide).

40. The method according to claim 9, wherein roflumilast represents a pharmaceutically acceptable salt of 3-cyclopropylmethoxy-4-difluoromethoxy-N-(3,5-dichloropyrid-4-yl)benzamide (ROFLUMILAST).

41. The method according to claim 9, wherein roflumilast represents a pharmaceutically acceptable salt of 3-cyclopropylmethoxy-4-difluoromethoxy-N-(3,5-dichloro-1-oxido-pyrid-4-yl)benzamide (ROFLUMILAST-N-Oxide).

42. The method according to claim 15, wherein the daily therapeutically effective amount of 3-cyclopropylmethoxy-N-(3,5-dichloropyrid-4-yl)benzamide (ROFLUMILAST) for an adult patient is 500 μg.

43. The method according to claim 16, wherein the daily therapeutically effective amount of 3-cyclopropylmethoxy-N-(3,5-dichloropyrid-4-yl)benzamide (ROFLUMILAST) for an adult patient is 500 μg.

44. The method according to claim 16, wherein the daily therapeutically effective amount of 3-cyclopropylmethoxy-N-(3,5-dichloro-1-oxido-pyrid-4-yl)benzamide (ROFLUMILAST-N-Oxide) for an adult patient is 500 μg.

45. The method according to claim 17, wherein the daily therapeutically effective amount of 3-cyclopropylmethoxy-N-(3,5-dichloro-1-oxido-pyrid-4-yl)benzamide (ROFLUMILAST-N-Oxide) for an adult patient is 500 μg.
